(19) United States
(12) Patent Application Publication
(10) Pub. No.: US 2006/0009404 A1
(43) Pub. Date: Jan. 12, 2006

(54) Tumor ablation in combination with pharmaceutical compositions

(76) Inventor: Jason R. Williams, Orange Beach, AL (US)

Correspondence Address: MATTHEW E. BURR;LAKE AUSTIN MARINA, 2219 WESTLAKE DR, STE 200, AUSTIN, TX, 78746, US

(21) Appl. No.: 10/888,433

(22) Filed: Jul. 9, 2004

(51) Int. Cl. A61K 48/00

(52) U.S. Cl. 514/44

(57) ABSTRACT

Therapeutic methods, compositions, and apparatus are provided for the treatment and ablation of body masses, such as tumors. RF treatment is combined with pharmaceutical compositions so that tumor masses are ablated and therapeutically effective compositions and doses that include or produce p53 tumor suppressor or peptides and variants thereof are administered to manage recurrence and metastasis.

### FIELD OF THE INVENTION

This invention relates generally to therapeutic methods, compositions, and apparatus for the treatment and ablation of body masses, such as tumors, and more particularly, to combinations of RF treatment and pharmaceutical compositions that combine tumor ablation with therapeutically effective compositions and doses that include or produce p53 tumor suppressor or peptides and variants thereof.

### BACKGROUND OF THE INVENTION

The present invention combines an initial course of minimally invasive tumor ablation to eliminate the great mass of a solid tumor, with therapeutic agents which, through their antiproliferative and/or apoptotic properties, interfere with hyperproliferative cell dysfunctions at the margins of the ablation locus, to reduce the ability of an aggressive cancer to re-assert itself.

1. Tumor Ablation

Many procedures for the treatment of tumors are extremely disruptive and cause a great deal of damage to healthy tissue. During a surgical procedure, for example, the physician must exercise care in cutting the tumor in a manor that does not seed the tumor, resulting in metastasis. The development, therefore, of products to minimize the traumatic nature of invasive surgical procedures is always welcome.

There has been a relatively significant amount of activity in the area of hyperthermia as a tool for treatment of tumors. It is known that elevating the temperature of tumors is helpful in the treatment and management of cancerous tissues. The mechanisms of selective cancer cell eradication by hyperthermia are not completely understood. However, four cellular effects of hyperthermia on cancerous tissue have been proposed, (i) changes in cell or nuclear membrane permeability or fluidity, (ii) cytoplasmic lysomal disintegration, causing release of digestive enzymes, (iii) protein thermal damage affecting cell respiration and the synthesis of DNA or RNA and (iv) potential excitation of immunologic systems. Treatment methods for applying heat to tumors include the use of direct contact radio-frequency (RF) applicators, microwave radiation, inductively coupled RF fields, ultrasound, and a variety of simple thermal conduction techniques.

Among the problems associated with all of these procedures is the requirement that highly localized heat be produced at depths of several centimeters beneath the surface of the skin. Certain techniques have been developed with microwave radiation and ultrasound to focus energy at various desired depths. RF applications may be used at depth during surgery. However, the extent of localization is generally poor, with the result that healthy tissue may be harmed. Induction heating gives rise to poor localization of the incident energy as well. Although induction heating may be achieved by placing an antenna on the surface of the body, superficial eddy currents are generated in the immediate vicinity of the antenna, when it is driven using RF current, and unwanted surface heating occurs with little heat delivered to the underlying tissue. Thus, non-invasive procedures for providing heat to internal tumors have had difficulties in achieving substantial specific and selective treatment.

Hyperthermia, which can be produced from an RF or microwave source, applies heat to tissue, but does not exceed 45 degrees C., so that normal cells survive. In thermotherapy, heat energy of greater than 45 degrees C. is applied resulting in histological damage, desiccation and the denaturization of proteins. Hyperthermia has been applied more recently for therapy of malignant tumors. In hyperthermia, it is desirable to induce a state of hyperthermia that is localized by interstitial current heating to a specific area while concurrently insuring minimum thermal damage to healthy surrounding tissue. Often, the tumor is located subcutaneously and addressing the tumor requires surgery, endoscopic procedures, or external radiation. It is difficult to externally induce hyperthermia in deep body tissue because current density is diluted due to its absorption by healthy tissue. Additionally, a portion of the RF energy is reflected at the muscle/fat and bone interfaces, which adds to the problem of depositing a known quantity of energy directly on a small tumor.

Attempts to use interstitial local hyperthermia have not proven to be very successful. Results have often produced non-uniform temperatures throughout the tumor. It is believed that tumor mass reduction by hyperthermia is related to thermal dose. Thermal dose is the minimum effective temperature applied throughout the tumor mass for a defined period of time. Because blood flow is the major mechanism of heat loss for tumors being heated, and blood flow varies throughout the tumor, more even heating of tumor tissue is needed to ensure effective treatment.

The same is true for ablation of the tumor itself through the use of RF energy. Different methods have been utilized for the RF ablation of masses such as tumors. Instead of heating the tumor it is ablated through the application of energy. This process has been difficult to achieve due to a variety of factors including, (i) positioning of the RF ablation electrodes to effectively ablate all of the mass, (ii) introduction of the RF ablation electrodes to the tumor site and (iii) controlled delivery and monitoring of RF energy to achieve successful ablation without damage to non-tumor tissue.

There have been a number of different treatment methods and devices for minimally invasively treating tumors. One such example is an endoscope that produces PF hyperthermia in tumors, as disclosed in U.S. Pat. No. 4,920,978. A microwave endoscope device is described in U.S. Pat. No. 4,409,993. In U.S. Pat. No. 4,920,978, an endoscope for RF hyperthermia is disclosed.

In U.S. Pat. No. 4,763,671 (the “'671 patent”), a minimally invasive procedure utilizes two catheters that are inserted interstitially into the tumor. The catheter includes a hard plastic support member. Around the support member is a conductor in the form of an open mesh. A layer of insulation is secured to the conductor with adhesive beads. It covers the entire conductor except a preselected length that is not adjustable. Different size tumors cannot be treated with the same electrode. A tubular sleeve is introduced into the support member and houses radioactive seeds. The device of the '671 patent fails to provide for the introduction of a fluidics medium, such as a chemotherapeutic agent, to the tumor for improved treatment. The size of the electrode conductive surface is not variable. Additionally, the device of the '671 patent is not capable of maintaining a preselected level of power that is independent of changes in voltage or current.

In U.S. Pat. No. 4,565,200 (the “'200 patent”), an electrode system is described in which a single entrance tract cannula is used to introduce an electrode into a selected body site. The device of the '200 patent is limited in that the single entrance tract fails to provide for the introduction, and removal of a variety of inserts, including but not limited to an introducer, fluid infusion device and insulation sleeve. Additionally, the device of the '200 patent fails to provide for the maintenance of a selected power independent of changes in current or voltage.

Heat has been used in medicine as long as history. Ancient Hindu medicine used heated metal bars and the Greeks used heated stones to stop bleeding. Electrocautery has been used for decades in surgery to fulgurate, cauterize, cut tissue, and to stop bleeding. The RFA generator uses a slight modification of the old technology to deposit the energy over a larger volume. The RFA generator also cauterizes tissue as it heats it, thus limiting blood loss and decreasing the risk of bleeding.

Percutaneous, minimally invasive, local treatment is an attractive new tool for the cancer patient, especially for disease in the liver. There is no existing effective treatment for the vast majority of patients with liver metastases. Most primary liver tumors are unresectable at the time of discovery. Historically, recurrence is common, even in candidates undergoing curative resection. Local treatment preserves uninvolved liver tissue, has potentially fewer systemic complications and side effects than systemic treatment options like chemotherapy, and avoids the morbidity and mortality of major liver surgery. RFA is fast, easy, predictable, safe, and relatively cheap. A multidisciplinary team approach is recommended to take care of the oncology patient.

Even minimally invasive procedures, however, such as RFA, invite the risk of tumor seeding. In particular, withdrawal of the ablation instrument from the body of the patient has been known to seed microscopic tumors along the insertion path. Furthermore, relocation of the of the ablation tip during the procedure, or due to a variety of factors, microscopic, invisible tumors or transformed cells that escaped ablation may cause the cancer to reassert itself sometime later. To mitigate the harm of tumor seeding from RFA, the present invention provides methods and compositions to treat the tumor site and the insertion path with tumor suppressing agents subsequent to ablation. An example of a tumor suppressor is p53, which is described in more detail below.

2. p53 Tumor Suppression

Compositions having proteins and peptides derived from the product of the tumor suppressor gene p53, have been demonstrated to elicit tumor suppression and programmed cell death. Such therapeutic effects are useful in pathological situations of proliferation in which the wild-type p53 protein is inactivated or impaired. The invention adapts compositions to the restore the functions of p53 in pathological situations such as cancers, administered in conjunction with tumor ablation.

Wild-type p53 protein is involved in regulating the cell cycle and in maintaining the integrity of the cell genome. Its main function is to activate the transcription of genes to initiate a cascade of DNA repair processes upon the appearance of mutations during the replication of the genome. Furthermore, in the event of a malfunctioning of these repair processes or in the event of the appearance of mutation events that are too many to be corrected, p53 induces programmed cell death, called apoptosis. p53 acts as a tumor suppressor by eliminating abnormally differentiated cells or cells whose genome has been damaged.

This principal function of p53 is as transcription factor. It recognizes specific sequences at the level of the genomic DNA and recruits the general transcription machinery.

The p53 protein comprises 393 amino acids that define 5 functional domains. The transcription-activating domain consists of amino acids 1 to 73 and is capable of binding factors of the general transcription machinery such as the TBP protein. Certain post-translational modifications occur at this domain. Numerous other proteins interact with p53 at this domain. For example, the cellular protein MDM2, or the protein EBNA5 of the Epstein-Barr virus (EBV), interact with p53 and are capable of blocking the function of the wild-type protein. Additionally, the domain has amino acid sequences termed PEST for susceptibility to proteolytic degradation.

The DNA-binding domain is located between amino acids 73 and 315. The conformation of this central domain of p53 regulates the recognition of DNA sequences specific for the p53 protein. It is the locus of certain alterations that affect the function of the wild-type protein. Interaction with proteins blocking the function of p53, such as the “large T” antigen of the SV40 virus or the E6 viral proteins of the HPV16 and HPV18 viruses cause its degradation by the ubiquitin system in the presence of the cellular protein E6ap (enzyme E3 of the ubiquitinilation cascade). Point mutations that affect the function of p53, are practically all located in this region. The nuclear localization signal consisting of amino acids 315 to 325 and essential for the correct directing of the protein in the compartment where it will exert its principal function.

Amino acids 325 to 355 constitute the oligomerization domain. This region forms a structure of the type: betasheet (326-334), elbow (335-336), and alpha helix (337-355). Interaction of the wild-type protein with the various mutant forms of this region alters the functions of the wild-type protein.

The regulatory domain, amino acids 365 to 393, is the locus of a number of post-translational modifications such as glycosylations, phosphorylations, attachment of RNA, and the like. Modifications modulate the function of the p53 protein in a positive or negative manner. The domain plays an extremely important role in the modulation of the activity of the wild-type protein.

The function of the p53 protein can be disrupted in various ways. One way is to block its function by a number of factors such as, for example, the “large T” antigen of the SV40 virus, the EBNA5 protein of the Epstein-Barr virus, or the cellular protein MDM2. Another way is to destabilize the protein by increasing its susceptibility to proteolysis. Interaction with the E6 protein of the human papilloma viruses HPV16 and HPV18, which promotes the entry of p53 into the ubiguitinilation cycle, is a effective method of destabilization. Other methods include point mutations at the level of the p53 gene and deletion of one or both p53 alleles.

The latter two types of modifications are found in about 50% of the various types of cancer. Mutations of the p53 gene in cancer cells affect a very large portion of the gene encoding the protein. The mutations result in varying modifications of its functionality. The great majority of these mutations are located in the central part of the p53 protein, which is known to be the region of contact with the genomic sequences specific for the p53 protein.

Most mutants of the p53 protein are unable to attach to the DNA sequences recognized by the wild-type protein. The mutants do not perform their function as transcription factor. Indeed, some mutants acquire new functions, such as the activation of genes at the transcriptional level. The mutations or modifications are currently classified into three categories.

(1) The so-called weak mutants, in which the product is a nonfunctional protein. In the case of a mutation on only one of the two alleles, a weak mutation does not affect the function of the wild-type protein encoded by the other allele. The principal representatives of this category are the H273 and W248 mutants, the latter being specific for the familial Li-Fraumeni syndrome for hypersensitivity to cancerous conditions.

(2) The dominant-negative mutants, in which the product is also a nonfunctional protein. In the case of a mutation on only one of the two alleles, and through interaction with the wild-type protein, these mutations block the function of the latter by forming non-active mixed oligomers that can no longer attach to the DNA sequences specific for the wild-type protein. The main representative of this category is the G281 mutant.

(3) The dominant-oncogenic mutants, in which the product is a protein that is capable, on the one hand, of blocking the function of the wild-type protein like the mutants of the previous category and, on the other hand, of promoting tumor development through poorly understood mechanisms. The principal representative of this category is the H175 mutant.

Taking into account its antitumor and apoptotic properties and its involvement in numerous pathologies of the hyperproliferative type, the wild-type p53 gene has been used in gene and cell therapy procedures. Clinical trials in various stages are underway to treat certain hyperproliferative pathologies, and especially cancers, by in vivo administration of the wild-type p53 gene and by restoring the functions of p53. The administration may be preferably carried out by viral and especially adenoviral (WO 94/24297) or retroviral (WO 94/06910) vectors.

The introduction of a nucleic acid encoding the wild-type p53 protein partially restores normal regulation of cell growth. Variants of the p53 protein that are resistant to the dominant-negative effect of some mutants have been developed, or are under development. Such variants display promising activity in a cellular context, exhibiting one or two mutated alleles, which is the case for nearly 90% of p53-dependent human cancers.

In some variants, an equivalent domain having a specific oligomerization capacity replaces all or part of the natural oligomerization domain of the protein. In particular embodiments, an optimized artificial leucine zipper is provded to form a dimer. The molecules having such an artificial leucine zipper are particularly advantageous because they form oligomers only with other molecules carrying the same leucine zipper. They do not, therefore, form oligomers with the dominant-negative or oncogenic mutants of the p53 protein, which are capable of inactivating them. Neither do they form oligomers with other cellular proteins carrying oligomerization domains, which are also capable of inactivating them or of inducing undesirable effects. They can only form homo-oligomers and therefore possess a high selectivity to ensure better activity against hyperproliferative pathology.

Certain modified variants have potentially enhanced “killer” properties, such as arresting the cell cycle and apoptosis. The combination of modifications, including the presence of a selective oligomerization domain and an improved transactivating power by substitution of the domain of origin and by the presence of a histidine in 182, confers on the variants considerably improved therapeutic potentials. In addition, selected variants avoid the appearance of some (dominant-oncogenic) mutants. The gains in function of some mutants of p53 are still poorly defined both at the level of their mechanisms and at the level of the domains of the p53 protein which are involved. It is highly probable that some of these new functions will depend on effective therapeutic combination with some effector cellular partners.

The existence of foreign units in the various constructs of the invention (murine AS protein for example, artificial oligomerization domain, and the like) may trigger an immune reaction during the death of the transfected cells and the release, into the extracellular medium, of these various fragments, thus increasing the capacity of the immune system to combat tumor cells.

An antibody or a fragment or derivative of an antibody directed against peptide or nucleic acid complexes are included in particular embodiments of the invention. Antibody fragments or derivatives are, for example, the fragments Fab or F(ab)′2, the regions VH or VL of an antibody or alternatively single-chain antibodies (ScFv) comprising a VH region bound to a VL region by an arm. The construction of nucleic acid sequences encoding such antibodies modified according to the invention has been described for example in U.S. Pat. No. 4,946,778 or in applications WO 94/02610, WO 94/29446.

A construct according to the present invention comprises an ScFv directed against a mutant of the p53 protein. These mutants appear in the transformed cells and possess a transactivating domain. Their recruitment by a variant according to the invention creates a chimeric molecule that becomes selectively activated in transformed cells.

Variants are contemplated that have an enhanced affinity for the sequences specific for binding DNA. Such variants advantageously comprises modifications in the N-terminal part as so as to further improve its properties. The modifications advantageously comprise a deletion of all or part of the transactivating domain. Any heterologous transactivating domain can replace the deleted domain. Preferably the transactivating domain is derived from the protein VP16 or a protein domain capable of specifically binding a transactivator or a transactivating complex present in a transformed cell. In addition, the residue 182 of the p53 protein is advantageously replaced by a histidine.

The subject of the present invention is also any nucleic acid encoding a variant or a chimeric protein. The nucleic acid according to the invention may be a ribonucleic acid (RNA) or a deoxyribonucleic acid (DNA). Preferably, the nucleic acid according to the invention is a cDNA or an RNA. A complementary DNA (cDNA) may comprise one or more introns of the p53 gene, or may comprise an antisense nucleic acid sequence. It may be of human, animal, viral, synthetic or semi-synthetic origin.

The nucleic acid may be obtained in various ways, such as by chemical synthesis using, for example, a nucleic acid synthesizer. It may also be obtained by the screening of libraries by means of specific probes. It may also be obtained by a combination of techniques including the chemical modification (elongation, deletion, substitution and the like) of sequences screened from libraries. In general, the nucleic acids of the invention may be prepared according to any technique known to a person skilled in the art.

Nucleic acids can be used as therapeutic agents to produce, in cells, derivatives capable of destroying or of correcting cellular dysfunctions. The present invention also relates to any expression cassette, such as are known to those skilled in the art, which provides a nucleic acid, a promoter allowing its expression, and a signal for termination of transcription.

The promoter is advantageously chosen from promoters that are functional in mammalian, preferably human, cells. More preferably, the promoter allows the expression of a nucleic acid in a hyperproliferative cell (cancerous, restenosis and the like). Accordingly, various promoters known in art can be used, including the promoter of the p53 gene itself. It may also be regions of different origin (which are responsible for the expression of other proteins, or which are even synthetic). It may thus be any promoter or derived sequence stimulating or repressing the transcription of a gene in a specific manner or otherwise, inducible or otherwise, strong or weak.

The promoter sequences of eukaryotic or viral genes are particularly indicated. They may be, for example, promoter sequences derived from the genome of the target cell. Among eukaryotic promoters, ubiquitous promoters may be used, in particular the promoter of the genes for HPRT, PGK, alpha-actin, tubulin and the like. Promoters of the intermediate filaments (promoter of the genes for GFAP, desmin, vimentin, neurofilaments, keratin and the like), and promoters of therapeutic genes (for example the promoter of the genes for MDR, CFTR, Factor VIII, ApoAI, and the like), are also suitable. Addiitionally, tissue-specific promoters (promoter of the gene for pyruvate kinase, villin, fatty acid-binding intestinal protein, smooth muscle a-actin and the like) or, alternatively, promoters responding to a stimulus (receptor for the steroid hormones, receptor for retinoic acid and the like) are candidate promoters of the present invention.

The promoter sequences may be derived from the genome of a virus, such as for example the promoters of the adenovirus EIA and MLP genes, the CMV early promoter, or alternatively the RSV LTR promoter and the like. Promoter regions modified by the addition of activation or regulatory sequences allow a tissue-specific or predominant expression.

### BRIEF DESCRIPTION OF THE DRAWINGS

The present invention is further described in the detailed description that follows, by reference to the noted drawing, by way of non-limiting examples of embodiments of the present invention, in which like reference numerals represent similar parts throughout several views of the drawings, and in which:

FIG. 1 is a schematic drawing of a multi-tined RFA instrument embodiment of the present invention inserted in a target tissue.

FIG. 2 is a schematic drawing depicting the tines of the embodiment of FIG. 1 detached from the RFA instrument in the target tissue.

FIG. 3 is an axial cross-section of an RFA instrument of one embodiment of the present invention.

FIG. 4 is a schematic side-view drawing of an RFA instrument of the present invention.

FIG. 5 is a schematic block diagram of the genome of P53.

### DETAILED DESCRIPTION

In view of the foregoing, the present invention, through one or more of its various aspects, embodiments and/or specific features or sub-components, is thus intended to bring out one or more of the advantages that will be evident from the description. The present invention is described with frequent reference to radio frequency ablation and p53. It is understood that radio frequency and p53 are merely examples of a specific embodiment of the present invention, which is directed generically to the therapeutic combination of ablation and tumor suppression compositions within the scope of the invention. The terminology, therefore, is not intended to limit the scope of the invention.

Cancer has traditionally been approached either systemically with chemotherapy, or locally with surgery or radiotherapy. Recent advancements in minimally invasive therapies are adding another tool to the anti-cancer arsenal. Thermal ablation is heating tumors so hot that the tumor cells die. It has been studied in many forms, including microwave, laser, high-intensity focused ultrasound, and cryotherapy. Radiofrequency thermal ablation or radiofrequency ablation (RFA) has emerged as the safest, easiest, and most predictable technology used for thermal ablation in the bone, liver, kidney, heart, prostate, breast, brain lymph nodes, nerve ganglia, and soft tissue.

Radiofrequency (RF) ablation is a technique for treating tumors localized to certain organs such as the adrenal glands, lungs, liver, and kidney. Relatively small probes are placed into the tumor and RF energy is transmitted through the probes. The RF energy causes the tissue around the tip of the probe to heat up to a high temperature above which cells break apart and die. RF kills both tumor and non-tumor cells. The probes are positioned so that they destroy the entire tumor plus an adequate “rim” of non-tumorous tissue around it.

Recent developments in radiofrequency ablation technology make large-volume tissue ablation (or cooking tumors) effective for local control of some cancer. Local tumor control is an attractive option for some patients who are not ideal surgical candidates, have failed conventional therapies, or have contraindications to surgery or recurrent tumors. Radiofrequency ablation may also expand surgical options. For example, RFA may convert an inoperable patient into a surgical candidate by treating small liver lesions that are too difficult or too spread out to remove with surgery.

Needle-based tissue ablation techniques performed through the skin may provide alternatives to open surgical procedures in certain patients, and may augment conventional therapies. Results suggest that RFA provides safe and effective local treatment of some cancers, with very small complication rates and preliminary survival curves similar to surgery for colorectal carcinoma liver metastases <4 cm, and hepatocellular carcinomas <5 cm. RFA provides a palliative treatment option for incurable disease, and appears to allow an increase in the rate of curative liver resection.

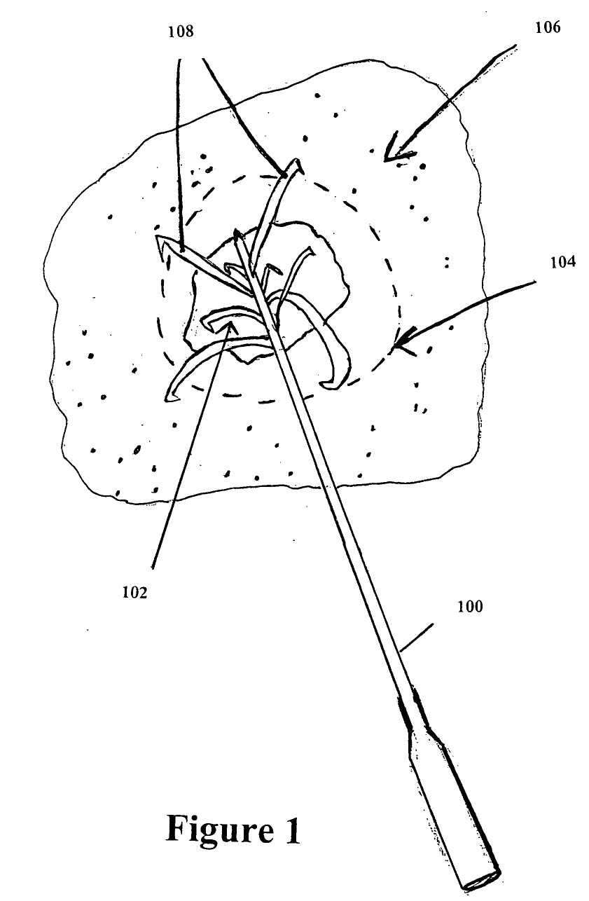

FIG. 1 is a schematic drawing of a multi-tined RFA instrument 100 of the present invention inserted in a target tissue 102, such as a solid tumor. Healthy tissue around the target, an ablation margin 104, is abated to better ensure complete treatment of the target. Tissue in thermal zone 106, around ablation margin 104, may heat up but is not killed.

Specific embodiments of the ablation instrument 100 provide a single ablation tine that applies heat to the target, but other embodiments, such as depicted in FIG. 1, provide multiple tines 108 that radiate from the central axis of the instrument tip to improve ablation of the target. Specific embodiments of the invention provide tines 108 to deliver pharmaceutical compositions of the invention as described below. Alternative embodiments provide detachable tines that remain in the target tissue after the instrument is withdrawn.

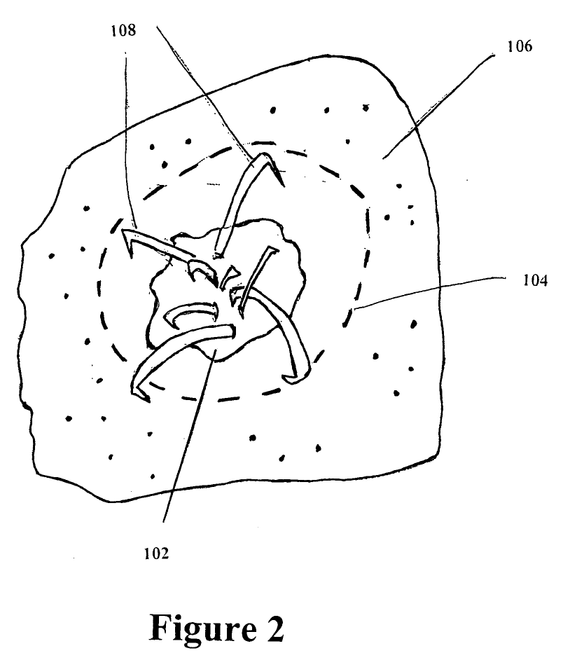

FIG. 2 is a schematic drawing depicting tines 108 of the embodiment of FIG. 1, detached from RFA instrument 100 in the target tissue 102 and within ablation margin 104. Another embodiment provides biodegradable detachable tines that non-toxically dissolve in the tissue, releasing an anti-tumor composition.

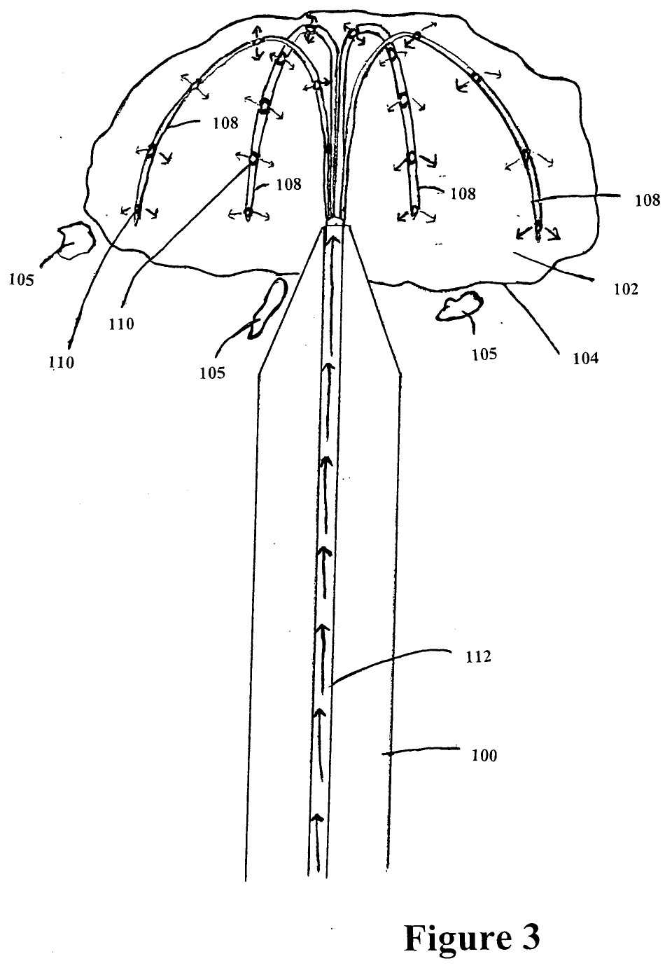

FIG. 3 is an axial cross-section of an alternative ablation instrument 100 of the present invention. The invention contemplates tines 108 having one or more conduit openings 110 from which a tumor suppressor composition of the invention infuses into the tissue. Conduit 112 delivers an anti-tumor pharmaceutical composition from a reservoir (not shown) to a plurality of tines 108, each tine 108 having a plurality of conduit openings 110 extending from the tip of instrument 100, from which the composition is expelled into the surrounding tissue 102. Pharmaceutical compositions for infusion are provided by injection, pumping, or even from passive fluid flow mechanisms such as by gravity or capillary action, from a reservoir through conduit 112 leading to tine openings 110 in certain embodiments. Alternatively, tines 108 are coated with a pharmaceutical composition that leaches or diffuses into the tissue.

Residual cancerous tissue 105, if any, is typically present around the margin of the ablated tissue and along the insertion bore of instrument 100. To mitigate the recurrence of cancer and prevent seeding transformed cells along the insertion bore, one or more anti-tumor compositions of the present invention are delivered by tines 108 to residual tissue 105 that escaped ablation.

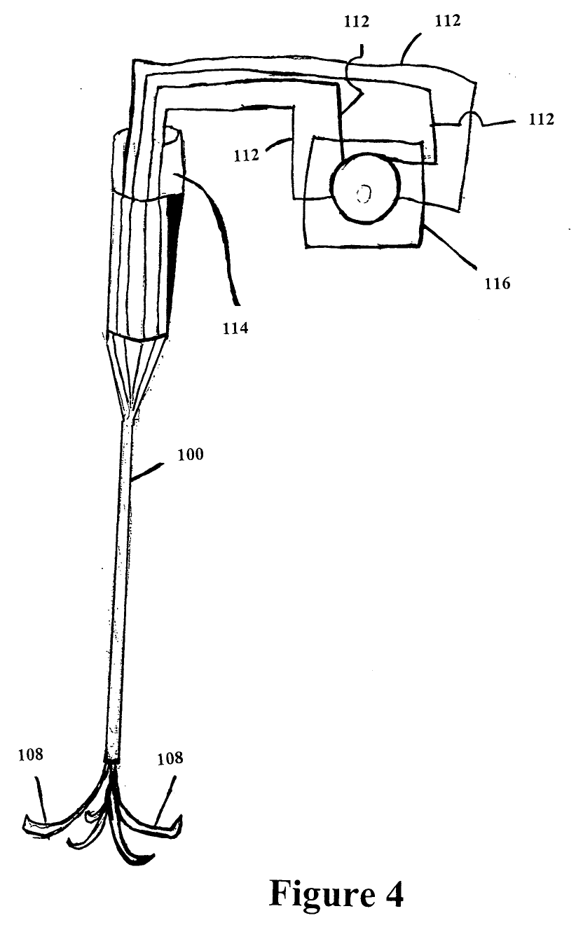

FIG. 4 is a schematic side-view drawing of RFA instrument 100 of the present invention. Tines 108 extend from the tip of instrument 100. Electric conductor leads 112 extend from each ablation tine 108 through handle 114 to RF or thermal generator 116.

RF ablation is often performed on an outpatient basis under general anesthesia or conscious sedation. The patient is made into an electrical circuit by placing grounding pads on the thighs. A 15 to 17.5-gauge needle-electrode with an insulated shaft and “hot” non-insulated tip, is inserted through the skin with imaging guidance using ultrasound, CT scan, or MRI. A treatment session has only 10 to 15 minutes of active ablation or cooking. The energy at the exposed tip causes ionic agitation and frictional heat, which cooks the tumor and leads to cell death and coagulative necrosis, if hot enough (above 50 degrees C.). Fibrosis and scar tissue gradually replace the necrotic tissue.

Over the subsequent months, the treated tissue shrinks in volume. Local recurrence, if any, occurs at the margin. Administration of an anti-tumor composition according to the present invention, however, mitigates the number of recurrence incidents and attenuates the aggressiveness of recurrent tumors.

The invention contemplates instrument 100 having an active tip of various lengths or configurations. The interventional radiologist of skill in the art uses knowledge of the underlying mechanism of thermal tissue ablation and the specific heat effects upon tissue to accurately predict ablation volume and shape, and to plan for disease-free treatment margins.

The procedure is usually performed by placing one or more probes through small (less than ¼ inch) incisions in the skin and using either ultrasound or a CT scanner to guide the tip into the tumor. For those tumors difficult to visualize by either US or CT, this procedure can also be performed in the operating room using a standard and much larger upper abdominal incision.

RF ablation is primarily used to treat liver tumors, either those that originate in the liver, such as hepatocellular carcinomas, or those that spread to the liver, such as metastatic disease. The technique has also been shown to be effective in treating tumors of the kidneys when surgery is not appropriate. There are also practitioners experienced in treating tumors in the adrenal gland and the lungs. However, most of long term data on RFA has been obtained from the treatment of liver tumors.

In patients with tumor isolated to their liver (no tumor in the lungs, lymph nodes, colon, etc.) improvements in survival have been noted. About a third of tumors demonstrate local recurrence although these areas can usually be retreated with RF ablation. Tumors adjacent to a major blood vessel often recur locally since the blood vessel itself draws heat away from the area during the treatment, the so-called “heat sink phenomenon”. As a result, the tumor cells next to the blood vessel cannot get hot enough to achieve cellular death.

The lesion to be treated is first localized by either CT or ultrasound. At times, both CT and ultrasound are used. A corresponding mark is made with a felt tip pen on the skin. The skin over the mark is then cleansed with a cold soap (Betadine) and a large plastic drape placed over it to maintain a sterile field.

Xylocalne, a local anesthetic similar to that used by dentists, is then introduced into the skin and soft tissue to numb these areas. There is a burning sensation for a few seconds. One to three tiny incisions, each measuring less than 5 mm in length, are then made in the skin.

The RF probe, which is similar in size to a biopsy needle, is then advanced into the lesion as guided by ultrasound, CT or both. Often, the probe has one or more tines. Once in place, the probe is hooked up to an electronic device and RF energy is applied to heat the tines for several minutes, depending upon the size of the lesion being treated. Larger lesions require longer or more treatment sessions. Since the objective is to destroy both the tumor and a cuff of normal tissue around the tumor, each lesion may be treated more than once.

After the treatments are finished, the needle is slowly withdrawn. Low power RF energy is also deposited along the needle tract upon withdrawal to minimize bleeding. After the procedure a band-aid is placed over the small incision(s). For lesions that are difficult to approach through the skin, this procedure can be performed in an open fashion in the operating room. That is, an incision is made in the upper abdomen, similar to that for a liver resection, and then the needle is inserted directly through the liver capsule into the lesion.

The application of RF energy into the body can be quite painful. Pain management options, therefore, are frequently available. One option is “conscious sedation”, whereby medications for pain and sedation are administered intravenously. The second option is “monitored anesthesia care” or MAC, whereby an anesthesiologist and/or anesthetist administer intravenous sedation. With MAC the level of anesthesia is generally deeper than it is with conscious sedation. The third option is a “general anesthesia”, which is also performed by an anesthesiologist and/or anesthetist and which is an even deeper level of sedation. This option requires placing a tube in the windpipe. For the first 12 hours after the procedure many patients experience only mild pain requiring an occasional Percocet tablet. Some have a bit more pain and require more Percocet for a longer period of time. A few patients have also experienced nausea for which we administer Phenergan either orally or intramuscularly.

Anytime a needle is placed under the skin there is almost always the risk of bleeding and infection. Bleeding complications are minimized by “coagulating” the tract with RF energy upon withdrawal of the probe. Administering antibiotics intravenously during the procedure minimizes infectious complications.

Other less common complications include diaphragmatic injury which often manifest as right shoulder pain, a skin injury when treating superficial lesions, and a collapsed lung for those lesions that are high under the diaphragm. The latter complication may require placement of a small tube between the lung and chest wall to reinflate the lung. Injury to other structures such as the bowels or blood vessels is unlikely when US or CT are used to guide probe placement. Experience has shown that all of these complications are uncommon, occurring in approximately 5% of patients or less.

Some lesions, particularly those that are larger, require more than one treatment session to destroy the entire tumor. In some patients, additional lesions will arise at a later date and these may also need to be retreated.

The present invention combines an initial course of minimally invasive tumor ablation to eliminate the great mass of a solid tumor, with therapeutic agents that, through their antiproliferative and/or apoptotic properties, interfere with hyperproliferative cell dysfunctions at the margins of the ablation locus, to reduce the ability of an aggressive cancer to re-assert itself.

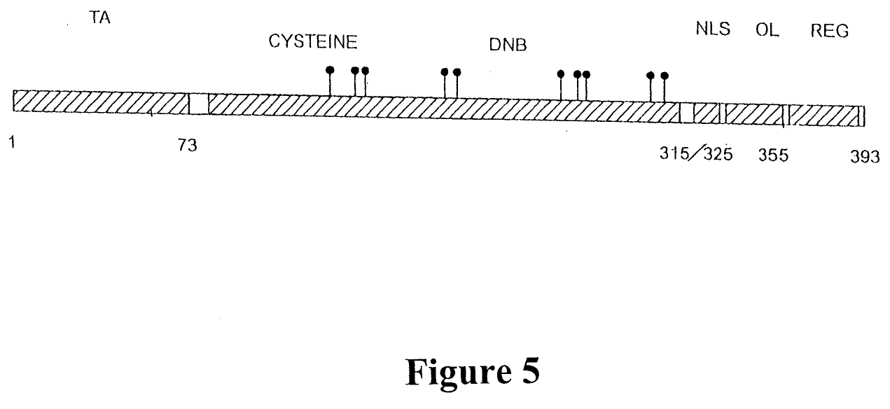

FIG. 5 is a schematic block diagram of the genome of P53. Nucleic acids or nucleic acid cassettes, as known in the art, providing pharmaceutical compositions of p53 of the present invention may be injected at the treatment site. Concurrently or alternatively, they may be incubated directly with the cells to be destroyed or to be treated. It has indeed been described that naked nucleic acids could penetrate into cells without any special vector. Nevertheless, it is preferred, within the framework of the present invention, to use a vector for administration which makes it possible to improve (i) the efficiency of cell penetration, (ii) the targeting, and (iii) the extra- and intracellular stability.

In a particularly preferred embodiment of the present invention, the nucleic acid or the cassette is incorporated into a vector. The vector used may be of chemical origin (liposome, nanoparticle, peptide complex, lipids or cationic polymers, and the like), or viral origin (retrovirus, adenovirus, herpes virus, AAV, vaccinia virus and the like) or of plasmid origin. The use of viral vectors rests on the natural transfection properties of viruses. It is thus possible to use, for example, adenoviruses, herpes viruses, retroviruses and adeno-associated viruses. These vectors are particularly efficient from the transfection standpoint. In this regard, a preferred subject according to the invention consists in a defective recombinant retrovirus whose genome comprises a nucleic acid as defined above. Another specific subject of the invention consists in a defective recombinant adenovirus whose genome comprises a nucleic acid as defined above.

The vector according to the invention may also be a nonviral agent capable of promoting the transfer and expression of nucleic acids in eukaryotic cells. Chemical or biochemical, synthetic or natural vectors represent an advantageous alternative to natural viruses in particular for reasons of convenience, safety and also by the absence of a theoretical limit as regards the size of the DNA to be transfected. These synthetic vectors have two principal functions, to compact the nucleic acid to be transfected and to promote its cellular attachment as well as its passage through the plasma membrane and, where appropriate, the two nuclear membranes. To overcome the polyanionic nature of nucleic acids, the nonviral vectors all possess polycationic charges.

The nucleic acid or vector used in the present invention may be formulated for topical, oral, parenteral, intranasal, intravenous, intramuscular, subcutaneous, intraocular and transdermal administration and the like. Preferably, the nucleic acid or vector is used in an injectable form. It may therefore be mixed with any vehicle, pharmaceutically acceptable for an injectable formulation, especially for a direct injection at the level of the site to be treated. This may be, in particular, sterile or isotonic solutions, or dry, especially freeze-dried, compositions that, upon addition, depending on the case, of sterilized water or of physiological saline, allow the preparation of injectable solutions. A direct injection of nucleic acid into the patient's tumor is advantageous because it makes it possible to concentrate the therapeutic effect at the level of the affected tissues. The doses of nucleic acid used may be adjusted according to various parameters, and especially according to the gene, vector, mode of administration used, pathology in question or alternatively the desired duration of treatment.

The invention also relates to any pharmaceutical composition comprising at least one nucleic acid as defined herein.

It also relates to any pharmaceutical composition comprising at least one vector as defined herein.

It also relates to any pharmaceutical composition comprising at least one variant of p53 as defined herein.

Due to their antiproliferative properties, the pharmaceutical compositions according to the invention are most particularly suitable for the treatment of hyperproliferative disorders, such as cancers and restenosis. The present invention thus provides an at least particularly effective method for the destruction of cells, especially of hyperproliferative cells in vivo. Administering to an organism an active quantity of a vector (or of a cassette) according to the invention, preferably directly at the level of the site to be treated (tumor in particular) inhibits the unregulated growth of tumor cells. A method of the invention, therefore, includes bringing hyperproliferative cells, or at least some of a population of such cells, into contact with a nucleic acid as defined above.

The present invention is advantageously used most appropriately for the treatment of cancers in which a mutant of p53 is observed. By way of example, there may be mentioned: colon adenocarcinomas, thyroid cancers, lung carcinomas, myeloid leukemias, colorectal cancers, breast cancers, lung cancers, gastric cancers, oesophageal cancers, B lymphomas, ovarian cancers, cancers of the bladder, glioblastomas, hepatocarcinomas, cancers of the bones, skin, pancreas or alternatively cancers of the kidney and of the prostate, oesophageal cancers, cancers of the larynx, head or neck cancers, HPV-positive anogenital cancers, EBV-positive cancers of the nasopharynx, cancers in which the cellular protein MDM2 is overexpressed, and the like.

The variants of the invention are particularly effective for the treatment of cancers in which the MDM2 protein is overexpressed, as well as cancers linked to the HPV virus, such as HPV-positive anogenital cancers. The term “variants” contemplates peptides derived from wild type p53, peptides derived from mutant or engineered (including chimeric) forms of p53, chemically or biologically modified p53 or peptides derived from p53, and mutant or engineered nucleic acids encoding p53 or domains thereof.

With respect to combination therapy of RFA with p53 (and variant) compositions, the present invention contemplates a variety of embodiments. For instance, certain RFA probes, such as provided by Rita Medical Systems, Inc., of Mountain view, Calif., USA, provide probes with tines, wherein each tine has conduit opening at the operational end of the tine. In one embodiment of the present invention, the conduits are adopted to deliver a selected pharmaceutical composition of the present invention either during the application of ablating RF, or subsequent to ablation of the tissue. The advantage being that concurrent or recent application of the pharmaceutical composition prevents, reduces or delays recurrence or regeneration of the cancer.

As with any invasive procedure, even minimally invasive ablation procedures, it is possible to disturb the tumor mass with the unwanted consequence of seeding, or potentially seeding, hyperproliferative cells away from the primary mass. Tumor seeding is pernicious, not the least because it is often undetected until a new mass appears, usually many months after treatment. An advantage of the present invention is that, in the event such seeding occurs, even undetected seeding, therapeutic dosing with one or more tumor suppressor compositions of the present invention prevents, reduces or delays recurrence or regeneration of the cancer that would otherwise be likely due to seeding.

In addition to pharmaceutical compositions of p53, the present invention also contemplates combinations of ablation treatment with one or more anti-angiogensis compositions. Another embodiment combines ablation with one or more anti-cancer vaccine compositions or regimens, such Gvax™. Other embodiments combine ablation with a pharmaceutical cocktail that includes, but is not necessarily limited to, for example, at least two compositions of the following: p53 or variants thereof, nucleotide sequences encoding for p53 or variants thereof, one or more anti-angiogenesis factors, one or more antisense oligonucleotide sequences, or one or more anti-cancer vaccine regimens. One or more kits are also contemplated to provide an RFA instrument together with one more pharmaceutical compositions of the invention.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses, either now known or which become known, such as are within the scope of the appended claims.

I claim:

1. A radio frequency ablation apparatus, the apparatus comprising an RFA needle having one or more tines, wherein at least one tine further comprises a pharmaceutical composition of p53.

2. The apparatus of claim 1, wherein the at least one tine comprises a conduit and a conduit opening to deliver the pharmaceutical composition.

3. The apparatus of claim 1, wherein the pharmaceutical composition comprises a nucleic acid sequence encoding p53.

4. The apparatus of claim 1, wherein the pharmaceutical composition comprises a peptide having a therapeutic amino acid sequence domain of p53.

5. The apparatus of claim 1, wherein the pharmaceutical composition comprises a variant of p53.

6. The apparatus of claim 1, wherein the pharmaceutical composition comprises a peptide having a therapeutic amino acid sequence domain of a p53 variant.

7. The apparatus of claim 3, wherein the nucleic acid sequence encodes a peptide that encompasses a therapeutic amino acid sequence domain of p53.

8. The apparatus of claim 3, wherein the nucleic acid sequence encodes a peptide that encompasses a therapeutic amino acid sequence domain of p53 variant.

9. A therapeutic method for the treatment of a solid tumor, the method comprising the steps of:
ablating at least a portion of the tumor; and
dosing the ablation site with a therapeutically effective dose and composition of p53.

10. A therapeutic method for the treatment of a solid tumor, the method comprising the steps of:
determining whether a tumor under-expresses p53;
selecting a tumor that under-expresses p53;
ablating at least a portion of the tumor; and
dosing the ablation site with a therapeutically effective dose and composition of p53.

11. A therapeutic method for the treatment of a solid tumor, the method comprising the steps of:
determining whether a tumor under-expresses p53;
selecting a tumor that under-expresses p53;
ablating at least a portion of the tumor; and
dosing the ablation site with a therapeutically effective dose and composition of a pharmaceutical cocktail, the cocktail comprising at least two of the group consisting of pharmaceutic compositions of:
p53 and therapeutic variants thereof, one or more nucleotide sequences encoding for p53 or therapeutic variants thereof;
one or more anti-cancer vaccine;
one or more antisense oligonucleotides, and therapeutic variants thereof, to one or more nucleic acid sense strands coding for p53 mutants, and
one more anti-angiogenesis factors.

12. The method of claim 11, further comprising the step of administering a GVAX regimen.

13. The method of claim 11, further comprising the step of inducing an anti-tumor immunological response.

14. A kit comprising an RFA instrument having one or more tines to deliver one or more anti-tumor pharmaceutical composition to tissue; and one or more anti-tumor pharmaceutical compositions for delivery to tissue by the RFA instrument.

15. The kit of claim 14, wherein at least one pharmaceutical composition induces an anti-angiogenesis response in the tissue.

16. The kit of claim 14, wherein at least one pharmaceutical composition induces an anti-tumor immunological response in the tissue.

17. The kit of claim 14, wherein at least one pharmaceutical composition induces a tumor suppression response in the tissue.

18. The kit of claim 17, wherein the pharmaceutical composition comprises a nucleic acid sequence encoding for a peptide that encompasses a therapeutic amino acid sequence domain of p53.
